SEQUENCE LISTING (8 sequences)

SEQ ID NO: 1 - DNA, 71 bp, Artificial Sequence
  aptamer
taccagtgcgatgctcaggccgatgtcaactttttctaactcactggttttgcctgacgcattcggttgac

SEQ ID NO: 2 - DNA, 71 bp, Artificial Sequence
  aptamer
taccagtgcgatgctcaggtggattgttgtgttctgttggtttttgtgttgtcctgacgcattcggttgac

SEQ ID NO: 3 - DNA, 71 bp, Artificial Sequence
  aptamer
taccagtgcgatgctcagccggggaccggggcaccgggggccagtggcacggactgacgcattcggttgac

SEQ ID NO: 4 - DNA, 71 bp, Artificial Sequence
  aptamer
gtcaaccgaatgcgtcagctggatcttaaagattgcatgcgctcactatgggactgagcatcgcactggta

SEQ ID NO: 5 - DNA, 47 bp, Artificial Sequence
  apatamer
accagtgcgatgctcaggccgatgtcaactttttctaactcactggt

SEQ ID NO: 6 - DNA, 71 bp, Artificial Sequence
  Library template
taccagtgcgatgctcagnnnnnnnnnnnnnnnnnnnnnnnnnnnnnnnnnnnctgacgcattcggttgac

SEQ ID NO: 7 - DNA, 18 bp, Artificial Sequence
  forward PCR primer
taccagtgcgatgctcag

SEQ ID NO: 8 - DNA, 18 bp, Artificial Seqence
  Reverse PCR primer
gtcaaccgaatgcgtcag

(19) United States
(12) Patent Application Publication
(10) Pub. No.: US 2009/0239762 A1
(43) Pub. Date: Sep. 24, 2009

(54) APTAMERS THAT BIND ABNORMAL CELLS

(76) Inventors: Weihong Tan, Gainesville, FL (US); Hul Chen, Gainesville, FL (US)

Correspondence Address: Aptomics, LLC, 12230 Forest Hill Boulevard, Ste 116, Wellington, FL, 33414, US

(21) Appl. No.: 12/366,460

(22) Filed: Feb. 5, 2009

Related U.S. Application Data
(60) Provisional application No. 61/063,640, filed on Feb. 5, 2008.

(51) Int. Cl. C40B 30/04; C07H 21/04; G01N 33/574; C12Q 1/68

(52) U.S. Cl. 506/9; 536/23.1; 435/7.23; 435/6

(57) ABSTRACT

A new aptamer approach for the recognition of specific small cell lung cancer (SCLC) cell surface molecular markers relies on cell-based systematic evolution of ligands by exponential enrichment (cell-SELEX) to evolve aptamers for whole live cells that express a variety of surface markers representing molecular differences among cancer cells. When applied to different lung cancer cells including those from patient samples, these aptamers bind to SCLC cells with high affinity and specificity in different assay formats. When conjugated with magnetic and fluorescent nanoparticles, the aptamer nano-conjugates could effectively extract SCLC cells from mixed cell media for isolation, enrichment, and sensitive detection.

### CROSS-REFERENCE TO RELATED APPLICATIONS

The present application claims the priority of U.S. provisional patent application Ser. No. 61/063,640 filed on Feb. 5, 2009 and entitled “Molecular Recognition of Small Cell Lung Cancer Cells Using Aptamers.”

### STATEMENT REGARDING FEDERALLY SPONSORED RESEARCH

The subject invention was made with government support under a research project supported by NIH National Institute of General Medical Sciences under Grant No. ROI GM079359.

### BACKGROUND OF INVENTION

A number of diseases are associated with the presence and/or proliferation of abnormal cells. Cancer, for example, is a leading cause of morbidity and mortality that can often be cured if diagnosed at an early stage. Among different types of neoplastic diseases, lung cancer is common and notoriously difficult to treat, accounting for 29% of all cancer deaths in the United States with a 5-year survival rate of less than 15%. A primary reason for the high death rate from lung cancer is that most lung cancer patients are diagnosed at an advanced stage when treatments are rarely successful.

Many different types of abnormal cells are known to contribute to disease. Differentiating among these abnormal cell types is often critical for correctly diagnosing and treating patients. For example, among all the lung cancer subtypes, small cell lung cancer (SCLC) has the highest tendency for early dissemination and the shortest median survival (7-12 months) as a clinically distinct entity. Survival of patients with SCLC therefore requires early detection as well as effective treatment. Advances in imaging-based screening technologies such as spiral computed tomography (CT), optical coherent tomography, positron emission tomography (PET), virtual bronchoscopy, autofluorescence bronchoscopy, and confocal microscopy has somewhat improved this situation, but unfortunately the morphological criteria used in imaging approaches are often not sufficiently sensitive enough to detect early stage disease. SCLC, for instance, can arise without morphologically recognizable preneoplastic lesions.

To improve this situation, molecular approaches were exploited for early detection of specific molecular markers. However, these molecular-marker based techniques also showed unsatisfactory results. For example, among more than 100 monoclonal antibodies for SCLC and non-small cell lung cancer (NSCLC), none of their antigens are exclusively expressed in SCLC samples. Therefore, the antibodies used for lung cancer early detection do not have the specificity, and may cross react with normal, mildly atypical, moderately atypical exfoliated epithelial cells, and even normal bronchial epithelium.

### SUMMARY

The invention is based on the development of nucleic acid based probes (aptamers) that preferentially bind a subset of abnormal cells using a technique called cell-SELEX (cell based systematic evolution of ligands by exponential enrichment). In a representative embodiment, aptamers that recognize lung cancer cells with sensitivity and selectivity were developed. When applied to different lung cancer cells, including those from patient samples, these aptamers bind to certain lung cancer cells with high affinity and specificity in a variety of assay formats. When conjugated with magnetic and fluorescent nanoparticles, these aptamer can be used to extract lung cancer cells from a biological sample. Thus, the invention can be used to detect, distinguish among, isolate, and enrich abnormal cells. The invention provides a means for accurately diagnosing the presence of abnormal cells that might contribute to the progression of diseases associated with abnormal cells (e.g., cancer).

Accordingly, the invention feature aptamers that specifically bind a lung cancer cell, and aptamers the bind to abnormal cells of a first type (e.g., small lung cancer cells) with greater affinity than to abnormal cells of a second type (e.g., non-small lung cancer cells). The aptamers can include a polynucleotide including the nucleic acid sequence of SEQ ID NO:1; SEQ ID NO:2; SEQ ID NO:3; SEQ ID NO:4; or SEQ ID NO:5. The aptamers of the invention can also be conjugated to another molecule such as a detectable label (e.g., a fluorophore or a radioisotope) or a nanoparticle.

In another aspect, the invention features a method of detecting an abnormal cell such as a lung cancer cell in a biological sample such as blood or sputum. This method includes the steps of: (a) providing a biological sample including an abnormal cell; (b) contacting the biological sample with an aptamer that selectively binds the abnormal cell; and (c) detecting the aptamer bound to the abnormal cell.

In a further aspect, the invention features a method including the steps of: (a) providing a single-stranded nucleic acid library including at least one million (e.g., at least 1×10⁶, 1×10⁷, 1×10⁸, 1×10⁹, or 1×10¹⁰) single-stranded nucleic acid molecules (e.g., DNA or RNA) having unique nucleic acid sequences; (b) providing a first sample of abnormal cells of a first type (e.g., SCLC cells); (c) mixing the library with the first sample under conditions which allow binding of some of the nucleic acid molecules in the library to the abnormal cells of the first type; (d) separating the nucleic acid molecules that bind to the abnormal cells of the first type from the nucleic acid molecules that do not bind to the abnormal cells of the first type; (e) mixing the separated nucleic acid molecules that bind to the abnormal cells of the first type with a first sample of abnormal cells of a second type (e.g., NSCLC cells) under conditions which allow binding of some of the separated nucleic acid molecules that bind to the abnormal cells of the first type to the abnormal cells of the second type; (f) separating the nucleic acid molecules that do not bind to the abnormal cells of the second type from the nucleic acid molecules that do bind to the abnormal cells of the second type; (g) collecting the nucleic acid molecules that do not bind to the abnormal cells of the second type, and optionally, (h) mixing the collected nucleic acid molecules that do not bind to the abnormal cells of the second type with a second sample of abnormal cells of the first type under conditions which allow binding of some of the collect nucleic acid molecules that do not bind to the abnormal cells of the second type to the abnormal cells of the first type; (i) separating the nucleic acid molecules that bind to the abnormal cells of the first type in step (h) from the nucleic acid molecules that do not bind to the abnormal cells of the first type; (j) mixing the separated nucleic acid molecules that bind to the abnormal cells of the first type of step (i) with a second sample of abnormal cells of the second type under conditions which allow binding of some of the separated nucleic acid molecules that bind to the abnormal cells of the first type of step (h) to the abnormal cells of the second type in the second sample; and (k) separating the nucleic acid molecules that do not bind to the abnormal cells of the second type in step (j) from the nucleic acid molecules that do bind to the abnormal cells of the second type; and (l) collecting the nucleic acid molecules that do not bind to the abnormal cells of the second type from step (k).

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994.

By “bind”, “binds”, or “reacts with” is meant that one molecule recognizes and adheres to a particular second molecule in a sample, but does not substantially recognize or adhere to other molecules in the sample. Generally, an aptamer that “specifically binds” another molecule has a K_{d} greater than about 10⁵, 10⁶, 10⁷, 10⁸, 10⁹, 10¹⁰, 10¹¹, or 1012 liters/mole for that other molecule.

The term “aptamer” is a nucleic acid macromolecule (e.g., DNA or RNA) that specifically binds to a molecular target by its tertiary conformation rather than by base pair complementarity.

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF DRAWINGS

FIG. 1 shows a scheme of Cell-SELEX for SCLC and Enrichment of Aptamers Along with the Progress of SELEX. FIG. 1A shows a number of DNA molecules from ssDNA library bind to SCLC cells after incubation, and are retained for counter-selection with NSCLC cells. The SCLC specific DNA molecules are subsequently PCR amplified for next round of selection, or for cloning and sequencing to identify individual aptamers in most selected pool. FIG. 1B shows a gradual evolution of SCLC specific aptamers along with the progress of SELEX. FITC-labeled ssDNA library and selected DNA pools were tested for binding to NCI-H69 (SCLC) and NCI-H661 (NSCLC) cells by flow cytometry. The binding ability of selected DNA pools gradually increased for SCLC, and no significant change was observed for NSCLC.

### DETAILED DESCRIPTION

The subject invention provides materials and methods for early diagnosis of pathological conditions such as lung cancer. Specifically, the subject invention provides molecular probes that recognize abnormal cells such as lung cancer cells with sensitivity and selectivity. In a specific embodiment, the subject invention provides an aptamer approach for the recognition of disease-associated cell markers such as specific SCLC cell surface molecular markers. The subject invention provides a new nucleic acid probe based approach for disease (e.g., cancer) diagnosis. One embodiment provides a panel of DNA aptamers that exploit the molecular differences among lung cancer cells in order to detect specific molecular markers on SCLC cell surfaces.

These aptamer probes were selected without prior knowledge about SCLC biomarkers. They were tested for their ability to specifically bind both cultured cells and clinical samples of SCLC in various assay formats. These aptamers can be used for detection and enrichment of SCLC cells, a critical step towards the goal of early detection where sensitive detection is needed.

Compared to other molecular recognition elements, the aptamers used in this approach present several advantages for early detection. While the aptamers' sensitivity leads to the detection of even small numbers of malignant cells, their specificity prevents cross reactivity with normal epithelial cells, resulting in fewer false positives. In addition, low-molecular weight aptamers can be easily synthesized and modified to recognize the target proteins at their native state on cell surfaces reproducibly.

The aptamers generated for certain SCLC cell lines are also able to recognize other SCLC cell lines of the same type, but seldom bind to other subtypes of lung cancer as well as other types of cancer (e.g., leukemia and liver cancer). Thus, the developed aptamer probes can be used reliably with clinical samples. In addition, the aptamers developed from live cells can also recognize fixed cells, the main assay format for retrospective analysis of preserved specimens in early detection study, as well as histological examination in clinical diagnosis of lung cancer. Notably, these aptamers exhibit the same specificity for cancer cells from SCLC patients as they do with cultured cells. In a complex biological environment such as human whole blood, this specific binding ability of aptamers was not compromised. Therefore, the developed aptamer probes are particularly well-suited for use in clinical tests.

The aptamers described herein were tested for possible application in early lung cancer detection, particularly enrichment and detection of exfoliated tumor cells, by using aptamer-conjugated magnetic nanoparticles and fluorescent nanoparticles. The high affinity and great specificity of these aptamers resulted in effective extraction of SCLC cells by magnetic separation, and the dye-doped nanoparticles gave rise to sensitive detection after cell extraction. Thus, the aptamer-conjugated nanoparticle strategy can be used to improve the efficiency of detecting circulating abnormal cells such as tumor cells; thereby providing a means for early detection of diseases such as lung cancer.

The aptamers described herein show great specificity for SCLC but not NSCLC. This is because these aptamers were generated based on the molecular differences between the two subtypes of lung cancer by cell-SELEX. The aptamers of the subject invention are suitable for multiple types of early detection studies. First, retrospective analysis of preserved specimens can be performed with these aptamers by using assay formats including flow cytometry and confocal imaging. Second, aptamer conjugated nanoparticles are able to isolate, enrich, and detect exfoliated tumor cells in peripheral blood. These aptamers can also be used for lung cancer subtyping during screening and planning appropriate treatment, for example, avoiding excessive therapy in the case of resectable NSCLC.

The combination of multiple markers facilitates enhanced accuracy compared to single markers used in previous studies. An additional notable advantage of this aptamer-based approach is that molecular markers are recognized at their native state on living cell surfaces. The molecular aptamers also may have important advantages over other methods for early lung cancer detection in terms of sensitivity, reproducibility, simplicity, robustness, production, and flexibility regarding modification. When coupled with appropriate assay formats, aptamers can be used in a variety of clinical tests.

This aptamer approach for early lung cancer early detection might also be able to detect pre-invasive lesions even before the malignant cells exfoliated when local therapy has limited effect, or indicate the possible relapse in early stage for proper therapy to prevent it if specific cell surface markers can be identified eventually. In addition, this approach can provide valuable information for the understanding of progressive neoplastic differentiation of lung cancer during early stages.

### General Aptamer Methods

General methods relating to aptamers are described in U.S. Pat. Nos. 5,270,163; 5,567,588; and 5,595,877. Aptamers are small single-stranded nucleic acid molecules approximately 10-120 nucleotides or 20-50 nucleotides in length that form secondary and/or tertiary structures which allows them to specifically bind to target molecules. Preferred aptamers of this invention are those that have high affinities, e.g., those with equilibrium dissociation constants ranging from 100 micromolar to sub-nanomolar depending on the selection used, and/or have high selectivity. Aptamers may be modified to improve binding specificity or stability as long as the aptamer retains a portion of its ability to bind and recognize its target monomer. For example, methods for modifying the bases and sugars of nucleotides are known in the art. Typically, phosphodiester linkages exist between the nucleotides of an RNA or DNA. An aptamer according to this invention may have phosphodiester, phosphoroamidite, phosphorothioate or other known linkages between its nucleotides to increase its stability provided that the linkage does not substantially interfere with the interaction of the aptamer with its target monomer.

Aptamers with improved characteristics (such as improved in vivo stability or improved delivery characteristics) can be prepared using techniques that are known to those of ordinary skill in the art. For example, chemical substitutions at the ribose and/or phosphate and/or base positions can be performed to improve aptamer stability in vivo. Additional techniques for improving aptamer characteristics include those described in U.S. Pat. No. 5,660,985; U.S. patent application Ser. No. 08/134,028; and U.S. patent application Ser. No. 08/264,029.

An aptamer suitable for use in the methods of this invention may be synthesized by a polymerase chain reaction (PCR), a DNA or RNA polymerase, a chemical reaction or a machine according to standard methods known in the art. For example, an aptamer may be synthesized by an automated DNA synthesizer from Applied Biosystems, Inc. (Foster City, Calif.) using standard chemical methods.

### Cell-SELEX

The subject invention provides another approach, the cell-SELEX approach, for identifying and isolating tumor-specific aptamers that are extremely useful in molecular profiling of targeted or diseased cells. Such a selection does not require prior knowledge of biomarker targets. The selection process is simple, reproducible, and straightforward. The aptamers of the invention can bind to target cells with Kd in the nM to pM range. Using the selected aptamers of the invention as molecular profilers for molecular profiling of cancer cells has yielded interesting information (such as regarding leukemia cells and normal human bone marrow aspirate). For example, some of the subject aptamers can only recognize a subset of the target cells, while others can bind to only one or two types of cancer cells. In addition, the isolation and identification of the target molecules recognized by these selected aptamers provide an effective and rapid way to discover disease biomarkers.

### Modified Aptamers

The aptamers of the invention can be modified by known methods. For example, a detectable label can be incorporated in or conjugated to an aptamer. The detectable label can be any molecule that can be detected by laboratory techniques, e.g., a dye, a fluorophore, a radioisotope, a particle (e.g., quantum dot or other nanoparticle), an enzyme, a magnetic agent, or a metal. The detectable label can be selected from many reactive fluorescent molecules that are known by and readily available to those of skill in the art. Specific labeled dyes that are useful in practicing the subject invention include, but are not limited to, dansyl, fluorescein, 8-anilino-1-napthalene sulfonate, pyrene, ethenoadenosine, ethidium bromide prollavine monosemicarbazide, p-terphenyl, 2,5-diphenyl-1,3,4-oxadiazole, 2,5-diphenyloxazole, p-bis[2-(5-phenyloxazolyl)]benzene, 1,4-bis-2-(4-methyl-5-phenyloxazolyl)benzene, and lanthanide chelate.

In certain embodiments, moieties such as enzymes, or other reagents, or pairs of reagents, that are sensitive to the conformational change of an aptamer binding to a target molecule, are incorporated into the engineered aptamers to form the aptamer probes. Such moieties can be incorporated into the aptamer either prior to transcription or post-transcriptionally, and can potentially be introduced either into known aptamers or into a pool of oligonucleotides from which the desired aptamers are be selected.

Upon binding of the aptamer probe to a target molecule, such moieties are activated and generate concomitant signals (for example, in the case of a fluorescent dye an alteration in fluorescence intensity, anisotropy, wavelength, or FRET). Such probes are particularly useful for clinical diagnosis of diseases (such as infections caused by organisms or cancer cells).

In other embodiments of the invention, moieties such as radioactive compounds or other known therapeutic compounds can be bound to the aptamer probe so as to provide treatment for the diseased cell. For example, a radioactive compound can be bound to an aptamer probe of the invention to act as an anti-bacterial, anti-viral and/or anti-fungal agent.

### Fluorophore Reporter Moieties

Fluorophore reporter moities can be, e.g., a fluorescence energy transfer pair that signals a conformation change in an aptamer probe, or conventional fluorescent labels whose efficiency is dependent on the conformation of the aptamer probe. Aptamer beacon reporter moieties can be a fluorophore and quencher or a charge or energy transfer system. A fluorophore can be 5-(2′-aminoethyl)aminoapthalene-1-sulfonic acid (“EDANS”), fluorescein, or anthranilamide. A quencher can be a chemical group, such as 4-(4′-dimethylaminophenylazo)benzoic acid (“DABCYL”), rhodamine, or cosine. A fluorophore and quencher can be incorporated into aptamer probes using techniques known in the art. See, e.g., Tyagi and Kramer, “Molecular Beacons: Probes That Fluoresce Upon Hybridization,” Nature Biotech., 14:303-08 (1996). The detectable moiety groups can also include an energy transfer system. An aptamer probe has an oligonucleotide with a binding region configured to bind a target molecule. The detectable moiety group includes an acceptor/fluorescence emitting moiety and a donor/energy absorbing moiety attached to oligonucleotide. When the emitting moiety and absorbing moiety are in proximity, energy transfers between the moieties to emit fluoresces efficiently. A fluorescence emitting moiety can be Cy5. An absorbing moiety can be fluorescein or tetramethyl rhodamine (“TMR”). The emitting moiety and absorbing moiety can be attached to oligonucleotides of the aptamer probe using techniques known in the art. See, e.g., Sixou et al., “Intracellular Oligonucleotide Hybridization Detected by Fluorescence Resonance Energy Transfer (FRET),” Nucleic Acids Res., 22:662-68 (1994).

Instead of designing aptamer probes with energy transfer reporters, other fluorescent reporters known in the art can be used. For example, an aptamer probe can be labeled with a fluorophore whose fluorescence efficiency depends on the environment (such as electrical, physical, or chemical environment) of the molecule to which it is attached. For example, binding of the target molecule to the aptamer-probe changes the conformation of the aptamer probe, thereby changing the chemical environment of the fluorophore, thereby causing a detectable change in the fluorescence of the fluorophore. Pyrene is a spatially sensitive fluorescent dye (see Fujimoto, K. et al. “Unambiguous detection of target DNAs by excimer-monomer switching molecular beacons.” Journal of Organic Chemistry, 69:3271-3275 (2004); Birks, J. B., Photophysics of Aromatic Molecules (Wiley Monographs in Chemical Physics) (1970); Winnik, F. M., “Photophysics of Preassociated Pyrenes in Aqueous Polymer-Solutions and in Other Organized Media,” Chemical Reviews, 93:587-614 (1993); and Lakowicz. J. R., Principles of Fluorescent Spectroscopy (Kluwer Academic/Plenum Publishers, New York, 1999)). Another example of a spatially sensitive fluorescent dye includes, but is not limited to, BODIPY Fl (see Dahim, M. et al, “Physical and photophysical characterization of a BODIPY phosphatidylcholine as a membrane probe,” Biophysical Journal, 83:1511-1524 (2002); and Pagano, R. E. et al, “A novel fluorescent ceramide analogue for studying membrane traffic in animal cells: accumulation at the Golgi apparatus results in altered spectral properties of the sphingolipid precursor,” Journal of Cell Biology, 113:1267-1279 (1991)). Both of these dyes, pyrene and BODIPY Fl, can form excited state dimers (excimers) upon close encounter of an excited state with another ground state molecule. The excimer emits at a longer wavelength than does a monomer.

An excimer is formed between two spatially sensitive fluorescent dyes (i.e., pyrenes) that are connected by a flexible covalent chain. As with FRET, the emission of the excimer is dependent upon the distance between the dyes. The stringent distance-dependent property of excimer formation is used in accordance with the subject invention as a unique means for signal transduction in the development of molecular probes. This is especially useful for developing aptamer probes due to the fact that many aptamers, like aptamers for PDGF-BB (see Fang, X. H., et al, “Molecular aptamer for real-time oncoprotein platelet-derived growth factor monitoring by fluorescence anisotropy,” Analytical Chemistry, 73:5752-5757 (2001); Nutiu, R. & Li, Y. F. “Structure-switching signaling aptamers: Transducing molecular recognition into fluorescence signaling,” Chemistry-A European Journal, 10:1868-1876 (2004); and Green, L. S. et al, “Inhibitory DNA ligands to platelet-derived growth factor B-chain,” Biochemistry, 35:14413-14424 (1996), cocaine (Stojanovic, M. N. et al., “Aptamer-Based Folding Fluorescent Sensor for Cocaine,” Journal of the American Chemical Society, 123:4928-4931 (2001)), and thrombin (Paborsky. L. R. et al., “The single-stranded DNA aptamer-binding site of human thrombin,” Journal of biological chemistry, 268:20808-20811 (1993); and Hamaguchi, N. et al., “Aptamer beacons for the direct detection of proteins,” Analytical Biochemistry, 294:126-131 (2001)) undergo conformation change upon target binding. In a preferred embodiment, the labeled dyes of the invention are attached to the terminal ends of the aptamer.

In one embodiment, the aptamer of the invention is labeled by preparing, purifying, and characterizing a manifold of derivatized, labeled nucleic acids. For example, a labeled dye is attached to a nucleic acid sequence, which serves as a primer for nucleic acid synthesis. A nucleic acid polymer is then annealed to the primer nucleic acid sequence to form an aptamer of the invention. Chemical methods are available to introduce fluorescence into specific nucleic acid bases by the reaction of chloroacetaldehyde with adenosine and cytidine to give fluorescent products. The reaction can be controlled with respect to which of the two bases is derivatized by manipulating the pH of the reaction mixture; the reaction at 37° C. proceeds rapidly at the optimum pH of 4.5 for adenosine and 3.5 for cytidine. See Barrio et al. Biochem. Biophys. Res. Commun. 46:597-604 (1972). This reaction is also useful for rendering fluorescent the deoxyribosyl derivatives of these bases. See Kochetkov et al., Dokl. Akad. Nauk. SSSR C 213:1327-1330 (1973).

In addition to the various methods for converting the bases of an intact aptamer into their fluorescent analogs, there are a number of methods for introducing fluorescence into an aptamer during its de novo synthesis. For example, a fluorescently tagged linker can be used that tethers an oligonucleotide strand to a solid support. When the oligonucleotide strand is cleaved from the solid support, the fluorescent tether remains attached to the oligonucleotide. This method affords an aptamer that is fluorescently labeled at its 3′-end. In a variation on this method, the 3′-end of the nucleic acid is labeled with a linker that bears an amine, or other reactive or masked reactive group, which can be coupled to a reactive fluorophore following cleavage of the oligonucleotide from the solid support. This method is particularly useful when the fluorophore is not stable to the cleavage or deprotection conditions. Another method relies on the selective labeling of the 5′ terminus of the oligonucleotide chain. Although many methods are known for labeling the 5′ terminus. the most versatile methods make use of phosphoramidites, which are derivatized with fluorophore or, if the fluorophore is unstable under the cleaving and deprotection conditions, a protected reactive functional group. The reactive functional group is labeled with a fluorophore following cleavage and deprotection of the oligonucleotide and deprotection of the reactive functional group.

### Use of Multiple Aptamers

In yet another aspect, the invention features a method or system for simultaneously detecting the presence or absence of one or more different target molecules in a sample using a plurality of different species of aptamer probes, wherein each species of aptamer probes has a different moiety or label dye group, a binding region that binds to a specific non-nucleic acid target molecule, and wherein the binding regions of different aptamers bind to different target molecules; and a detection system that detects the presence of target molecules bound to aptamer probes, the detection system being able to detect the different moiety or label dye groups. The method can also be carried out with a plurality of identical aptamer probes. For example, each aptamer can include a moiety such as a molecular beacon that changes fluorescence properties upon target binding. Each species of aptamer probe can be labeled with a different fluorescent dye to allow simultaneous detection of multiple target molecules, e.g., one species might be labeled with fluorescent and another with rhodamine. The fluorescence excitation wavelength (or spectrum) can be varied and/or the emission spectrum can be observed to simultaneously detect the presence of multiple targets.

### Target Molecules

The probes of the invention have the ability to interact with any target compound or cell (such as virus, bacteria, fungus, cancer). The subject invention utilizes the unique properties of aptamers to form probes for use in therapeutic practices, disease diagnosis and protein functional studies. These aptamers, which are integrated with a novel signal transduction mechanism, form sensitive and selective probes for use in protein detection. In one embodiment, the signal transduction mechanism is provided by spatially sensitive fluorescent dyes that form an excimer. The generation of the excimer emission requires the conformation change of the aptamer brought about by complexation with a target protein to bring two pyrene molecules together. This stringent requirement prevents false positive signals when the probe is digested by nucleases.

### EXAMPLES

The following examples illustrate procedures for practicing the invention. These examples should not be construed as limiting. All percentages are by weight and all solvent mixture proportions are by volume unless otherwise noted.

### Example 1

### Materials and Methods

Chemicals: Unless otherwise noted, all chemicals were purchased from Sigma-Aldrich and Fisher Scientific.

Buffers: Washing buffer was prepared by dissolving glucose (4.5 g/L), MgCl2 (5 mM), and BSA (1 mg/mL) in Dulbecco's PBS (pH 7.3). Yeast tRNA (0.1 mg/ml) was added in washing buffer to prepare binding buffer with minimal nonspecific binding.

Cell culture: NCI-H69 (small cell carcinoma), NCI-H661 (large cell carcinoma), NCI-H146 (small cell carcinoma), NCI-H128 (small cell carcinoma), NCI-H23 (adenocarcinoma), NCI-H1385 (squamous cell carcinoma), CCRF-CEM (T cell acute lymphoblastic leukemia), and Ramos (B cell human Burkitt's lymphoma) cells were purchased from American Type Culture Collection (ATCC), and maintained at 37° C. and 5% CO2 in RPMI 1640 medium (ATCC) supplemented with 10% heat-inactivated fetal bovine serum (FBS) (GIBCO) and 100 units/ml penicillin-streptomycin (Cellgro). IMEA (liver cancer) and BNL (liver cancer) cells were obtained from the Department of Pathology at the University of Florida.

DNA synthesis and purification: An ABI 3400 DNA Synthesizer (Applied Biosystems) was used for synthesis of single stranded DNA library (71 mer containing randomized 35 nucleotides and two primer binding sites, 5′-TACCAGTGCGATGCTCAG (N)35 CTGACGCATTCGGTTGAC-3′) [SEQ ID NO:6], PCR primers, and selected aptamers. The product was further purified by HPLC (Pro Star, Varian) using a C18 column (Econosil, 5U, 250×4.6 mm, Alltech Associates) and a linear elution gradient. The HPLC purified product was then dried, detrityled, and re-suspended in buffer for use. UV-Vis measurements were performed with a Cary Bio-300 UV spectrometer (Varian) for DNA quantitation.

Cell-SELEX: Target cell (NCI-H69) and control cell (NCI-H661) were counted and tested for viability before experiments. The ssDNA library (10 nmol in 1 mL binding buffer) was first denatured at 95° C. for 5 minutes and kept on ice for 10 minutes. 2×10⁶ target cells were washed, dissociated (0.53 mM EDTA/PBS), and then incubated with ssDNA library at 4° C. for 30 minutes. After washing, the cell bound DNAs were eluted to 300 μL binding buffer by heating at 95° C. for 5 minutes. The eluted DNAs were further incubated with excess control cells at 4° for 30 minutes for counter selection (eliminated in first round of selection). After counter selection, the DNAs that don't bind to control cells were collected, desalted, and PCR amplified with FITC and biotin labeled primers. The PCR product of first round of selection was then processed to generate single stranded DNAs for next round of selection. For the second round of selection, all product of first round was dissolved in 200 μL binding buffer as starting ssDNA pool. To increase the stringency of selection, the washing strength was enhanced by gradually increasing washing time (from 1 to 10 minutes), washing volume (from 1 to 3 mL), and washing round (from 3 to 5 times). The SELEX progress was monitored by flow cytometry.

Real-time PCR: At the end of every round of selection, target cell specific DNA molecules were PCR amplified to form the starting pool for next round of selection. Real-time PCR was first performed to determine the amount of DNA molecules to be amplified, using iTaq DNA polymerase (Bio-Rad) and a MyiQ real-time PCR system (Bio-Rad). SYBR green (Molecular Probes) was used for the detection of PCR products. PCR cycles were then optimized according to the template amount. The bulk of target cell specific DNA molecules was finally PCR amplified with the optimized PCR conditions. Primers for PCR amplification are:

| | |
|---|---|
| Forward primer | |
| 5′-TACCAGTGCGATGCTCAG-3′, | [SEQ ID NO: 7] |
| Reverse primer | |
| 5′ -GTCAACCGAATGCGTCAG-3′. | [SEQ ID NO: 8] |

Unlabeled forward and reverse primers are used for real-time PCR detection with SYBR green. FITC labeled forward primer and triple-biotinylated (trB) reverse primer are used to generate PCR product for flow cytometry assay. TAMRA labeled forward primer and triple-biotinylated (trB) reverse primer are used to generate PCR product for confocal imaging. PCR parameters consisted of 3 minutes of Taq activation at 95° C., and 15 cycles of PCR at 94° C. for 30 s, 52° C. for 30 s, 72° C. for 15 s, followed by 5 minutes of extension at 72° C. Standard curves were generated for real-time PCR. Specificity of PCR amplification was verified by melt curve analysis. Amplification products were also resolved by agarose gel electrophoresis and visualized by ethidium bromide staining.

Single-stranded DNA generation: To generate single stranded DNA from PCR product for next round of selection, the sense ssDNA was separated from the biotinylated anti-sense ssDNA by streptavidincoated sepharose beads (Amersham Pharmacia Biosciences). After elution with alkaline solution (0.2 M NaOH), the sense ssDNA was desalted with a Sephadex G-25 column (NAP-5, Amersham Pharmacia Biosciences), quantified by UV measurement, and dried in a SpeedVac. The product was then resuspended in buffer to be used for next round of selection.

Molecular cloning: To isolate individual aptamers from selected pool, cloning was performed after 25 rounds of selection. The most selected ssDNA pool was PCR amplified with unlabeled primers, and inserted into the pCR 2.1-TOPO TA Cloning vector (Invitrogen). The vector was then transformed into Escherichia coli. Cultured monocolonies were picked up to extract the plasmids for sequencing.

Sequencing: Cloned sequences were determined with 454 Life Sciences DNA sequencing unit, GS20, at Interdisciplinary Center for Biotechnology Research (ICBR) of the University of Florida.

Multiple sequence alignment analysis: The sequencing results were subjected to the multiple sequence alignment analysis with the MEME/MAST SYSTEM, version 3.5.3 (developed by Timothy Bailey, Charles Elkan, and Bill Noble at the UCSD Computer Science and Engineering department with input from Michael Gribskov at Purdue University, http://meme.nbcr.net) to discover highly conserved motifs in groups of selected DNA sequences. The discovered consensus sequences with high repeats among selected pool were then synthesized and tested for specificity and affinity.

Flow cytometry: To monitor the enrichment of aptamers along with the progress of SELEX, FITC labeled ssDNA pools were incubated with 1×106 NCI-H69 or NCI-H661 cells in 400 μL binding buffer at 4° C. for 30 minutes. Cells were washed twice after incubation and analyzed by flow cytometry. The binding of selected aptamers to SCLC cells, NSCLC cells, leukemia cells, and liver cancer cells were similarly analyzed. Flow cytometry was performed on a FACScan cytometer with CellQuest software (Becton Dickinson).

Confocal imaging: The binding of selected ssDNA pools and individual aptamers to SCLC cells was evaluated by fluorescence confocal imaging. Cells were incubated with 250 nM TAMRA labeled aptamers in 100 μL binding buffer at 4° C. for 30 minutes. After washing, 20 μL cell suspension was dropped on a covered glass slide for examination with confocal microscope. Fluorescence confocal imaging was performed on a Fluoview 500/IX81 inverted confocal scanning microscope system (Olympus). A 5-mW, 543-nm He—Ne laser was used as excitation source for TAMRA dye. The objective used for imaging was a 60× oil-immersion objective (PLAPO60XO3PH) with a numerical aperture of 1.40 (Olympus). A 20× objective with a numerical aperture of 0.7 (Olympus) was also used for imaging of large field. Staining of cell line tissue array by fluorescent aptamers and extraction of SCLC cells by aptamer conjugated nanoparticles were evaluated by confocal imaging as described above.

Saturation analysis: Saturation analysis was performed to measure the relative cell surface binding affinities of developed aptamers. Cells were incubated with FITC labeled aptamers at 4° C. for 30 minutes, washed three times with 400 μL washing buffer, and finally re-suspended in 400 μL binding buffer containing 20% FBS. Cells were then assayed using flow cytometry. Concentrations of FITC labeled aptamers for the relative affinity measurements varied from 0 to 1 μM. The FITC labeled ssDNA library was used to determine nonspecific binding. The mean fluorescence intensity of aptamer bound cells (nonspecific binding of DNA library subtracted) was used to calculate bound aptamer fraction at different concentrations. All affinity measurements were performed in triplicate. The results are described as mean±s.e.m. The equilibrium dissociation constants (Kd) were obtained by fitting the cell surface binding data of aptamers to a one-site saturation model with SigmaPlot 9.0 (Jandel Scientific).

Enzymatic treatment: To verify the binding of aptamers to SCLC cell surface markers, cells were examined by enzymatic treatment. 1×10⁶ Cells were washed with 1 ml of PBS, and treated with 200 μL of 0.05% trypsin/0.53 mM EDTA in HBSS (Fisher Biotech) or 0.1 mg/mL proteinase K (Fisher Biotech) in PBS at 37° C. for 2 minutes. FBS was then added to quench the enzyme activity. After washing with binding buffer, the cells were analyzed for aptamer binding with flow cytometry and confocal imaging as described above.

Cell line tissue array: Cultured SCLC and NSCLC cell lines were processed into homogeneous tissue arrays to evaluate the binding of aptamers to fixed cells. All cell line tissue arrays were prepared in the University of Florida Diagnostic Reference Laboratories. 10×106 cells grown in culture were first prepared as a cell suspension in minimal amount of medium (adherent cells were detached by trypsin EDTA treatment). Cells were then fixed with 4% formaldehyde, and mixed with 1% agarose in isoosmotic PBS. The solidified cell blocks were cut into serial sections and processed on paraffin-embedded slides. Prepared cell line tissue arrays were stained with hematoxylin and eosin (H&E) for quality control.

Cell line tissue array staining: Cell line tissue arrays were first treated with xylene and ethanol (100%, 95%, and 70%) for deparaffinization. For antigen retrieval, the dried tissue arrays were rinsed with PBS and kept in 1 mM EDTA Tris buffer (pH 8.0) at 9° C. for 15 minutes. Tissue arrays were then incubated with 200 μL of 0.25 μM TAMRA labeled aptamers in binding buffer at 4° C. for 30 minutes. After washing and dehydration, the stained array slides were mounted for evaluation. Aptamer staining of cell line tissue arrays were analyzed by array scanning and by confocal imaging. For the array scanning, the stained array slides were scanned into a computer with a microarray scanner (2100 BioAnalyzer, Agilent) at 10 μm scan resolution, and analyzed using Agilent G2567AA Feature Extraction software (v.9.1). To confirm the array scanning results and show the binding details, the same stained array slides were imaged using an FV500-IX81 confocal microscope (Olympus) with a 543-nm excitation source. Images were collected with both 60× and 20× objectives as described above.

Clinical sample test: SCLC patient samples were obtained from the Department of Pathology at the University of Florida. Cells were washed and counted for incubation with aptamers. Cell surface binding of FITC labeled aptamers was analyzed by flow cytometry as detailed above.

Binding assay in human whole blood: To evaluate the binding capacity of aptamers in complex biological environment, 2×10⁶ SCLC cells were prepared as detailed above and mixed with 3.5 μL human whole blood (IPLA-WB1, Innovative Research, Inc.) in 300 μL of buffer. Human whole blood was prepared by mixing with the anticoagulant, sodium heparin. 100 μL of 1 μM FITC labeled aptamers was added to SCLC cells previously spiked in human whole blood. After incubation at 4° C. and thorough washing, we assessed the binding of aptamers to SCLC cells in blood with flow cytometry. For controls, human whole blood and cells in buffer were incubated with aptamers and analyzed by flow cytometry. Background binding of aptamers to blood cells was negligible.

Aptamer conjugated magnetic and fluorescent nanoparticles: For the synthesis of aptamer conjugated magnetic nanoparticles, the 65-nm iron oxide-doped magnetic nanoparticles were first prepared by precipitating iron oxide. The magnetite core particles were then coated with silica by the hydrolysis of tetraethoxyorthosilicate, and treated with TEOS. After washing, avidin coating was performed by incubating 0.1 mg/mL silica-coated magnetic nanoparticle solution with 5 mg/mL avidin solution at 4° C. for 12 hours. The avidin-coated magnetic nanoparticles were then washed with PBS, and stabilized by crosslinking with 1% glutaraldehyde at 25° C. for 1 hour. After washing with Tris-HCl buffer, the 0.2 mg/mL avidin-coated magnetic nanoparticles were incubated with excess biotinylated DNA aptamers and ssDNA library at 4° C. for 12 hours. The prepared aptamer conjugated magnetic nanoparticles were washed and stored at a final concentration of 0.2 mg/mL at 4° C. for use.

For the synthesis of aptamer conjugated fluorescent nanoparticles, TAMRA dye-doped nanoparticles were first prepared by the reverse microemulsion method. After silica polymerization and stabilization treatment with TEOS, the dye-doped nanoparticles were coated with avidin as detailed above. Avidin coated dye-doped nanoparticles were further conjugated with excess biotinylated DNA aptamers and ssDNA library. The prepared aptamer conjugated fluorescent nanoparticles were washed and stored at a final concentration of 10 mg/mL at room temperature for use.

Extraction and detection of SCLC cells: For every experiment, 1.0×10⁵ cells were prepared as detailed above and dispersed in 200 μL of cell media buffer. The specified amount of aptamer conjugated magnetic and fluorescent nanoparticles was then simultaneously added to the cell suspension. After 30 minute incubation and washing, cells were isolated from cell media buffer by magnetic extraction, and recovered in 20 μL of buffer for confocal imaging and fluorescence measurement. A 2-μL aliquot of the extracted sample was assessed by confocal imaging as described above. The rest samples were then added to 96-well plate, and the fluorescence of dye-doped nanoparticles bound to extracted cells was measured by a plate reader (Packard). ssDNA library conjugated magnetic and fluorescent nanoparticles were used for control experiments.

### Example 2

### SELEX for Whole Live Cancer Cells

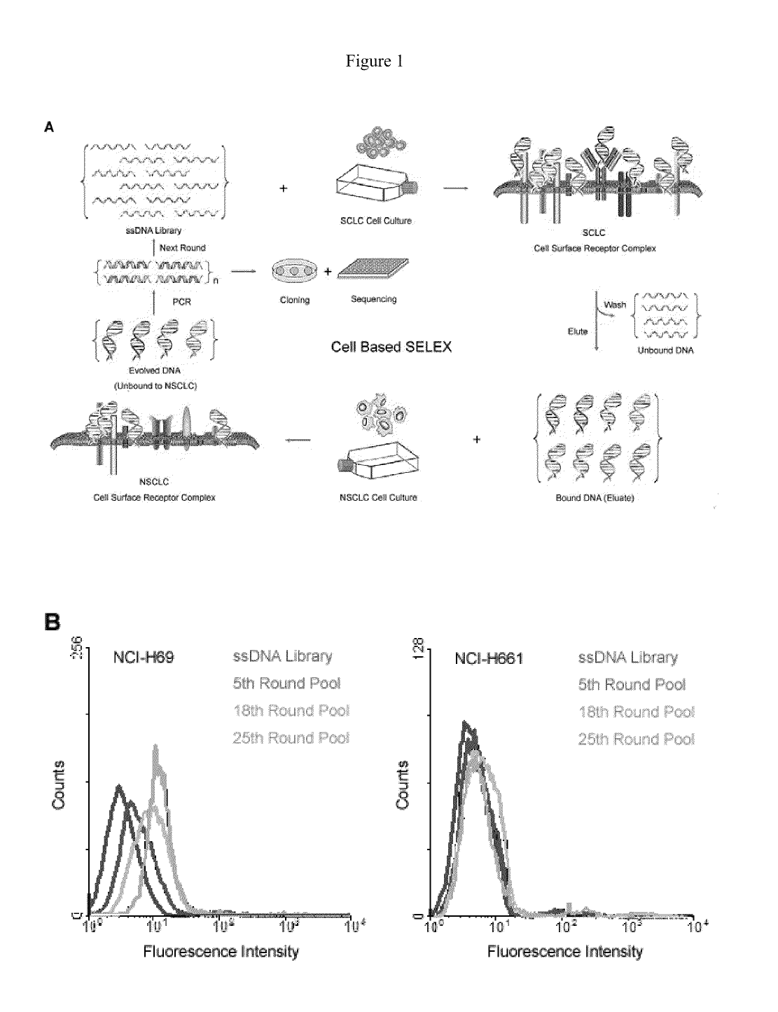

Referring to FIG. 1, to develop cell specific aptamer probes, live cancer cells were directly used as the target for cell-SELEX. This approach was adapted in a few aspects to work with floating aggregates of SCLC and adherent monolayers of NSCLC, which are two typical growth patterns of lung cancer culture. Because of their heterogeneity and poor viability, it is more challenging to perform cell-SELEX with lung cancer than leukemia. NSCLC (large cell) was adopted as a control for cell-SELEX to generate aptamers exclusive to the cell surface markers of SCLC. These cell surface markers are so exclusive to SCLC that normal lung epithelial cells are also not expected to bear them and cross-react with developed aptamers, as observed in previous studies with antibodies. With counter-selection against control cells, the aptamers achieve great selectivity necessary for the reliable detection of lung cancer antigens.

In the actual selection, a cultured SCLC cell line, NCI-H69, was first incubated with a 71-base synthetic single stranded DNA library. The DNA sequences that bound to target cells were then eluted after stringent washing. A cultured NSCLC cell line, NCI-H661, was introduced as control cell to separate aptamers with affinity to both the target and control cells from those aptamers recognizing only target cells in the previously eluted DNA pool. The remaining target cell specific sequences from counter-selection were further PCR amplified to form the starting pool of next round of selection. A panel of aptamer probes eventually evolved to have great specificity and high affinity for SCLC along with the SELEX progress.

The gradual enrichment of aptamers was monitored during the selection process by both flow cytometry and confocal microscopy. The ability of DNA pools from each round of selection to bind target cells was assessed. The increase in the fluorescence intensity of the dye labeled DNA pools bound to target cells is gradual and steady along with the progress of selection, indicating a successful evolution of high affinity aptamers. By contrast, no significant change was observed in the response to the control cells during the selection process, demonstrating the specificity of selected DNA pools.

After 25 rounds of selection, the binding ability of selected DNA pools reached a plateau, and cloning was performed to isolate individual aptamers in the most selected DNA pool. Results of subsequent sequencing were further analyzed by multiple sequence alignment software. The majority of aptamers in the selected pool belong to several families based on the consensus sequences.

To deconvolute the selected DNA pool, those consensus sequences with high repeats were synthesized to test their ability to specifically bind SCLC cells. A few of them showed prominent binding ability for SCLC but not NSCLC (control cell), as determined by flow cytometric analysis. The dominant peak refers to the binding of aptamer with SCLC cells. A second peak with high fluorescence signal was also noticed, which may have represented the population of dead cells. According to confocal imaging results, fluorescent dye-labeled aptamers specifically bound only to target SCLC cells. In addition, individual aptamers were tested with saturation analysis, and found to have high affinity with equilibrium dissociation constant in the nanomolar range (Table 1).

TABLE 1
Equilibrium Dissociation Constant of Selected SCLC Aptamers

| Selected sequence name | Kd |
|---|---|
| HCA12 | ~97 nM |
| HCC03 | ~123 nM |
| HCH07 | ~38 nM |
| HCH01 | ~157 nM |

Nucleic acid sequences of exemplary SCLC-specific Aptamers are listed below:

HCH07

[SEQ ID NO: 1]

TACCAGTGCGATGCTCAGGCCGATGTCAACTTTTTCTAACTCACTGGTTT
TGCCTGACGCATTCGGTTGAC
HCA12

[SEQ ID NO: 2]

TACCAGTGCGATGCTCAGGTGGATTGTTGTGTTCTGTTGGTTTTTGTGTT
GTCCTGACGCATTCGGTTGAC
HCC03

[SEQ ID NO: 3]

TACCAGTGCGATGCTCAGCCGGGGACCGGGGCACCGGGGGCCAGTGGCAC
GGACTGACGCATTCGGTTGAC
HCH01

[SEQ ID NO: 4]

GTCAACCGAATGCGTCAGCTGGATCTTAAAGATTGCATGCGCTCACTATG
GGACTGAGCATCGCACTGGTA
HCH07-47MER

[SEQ ID NO: 5]

ACCAGTGCGATGCTCAGGCCGATGTCAACTTTTTCTAACTCACTGGT

### Example 3

### Enzymatic Treatment of Cell Surface Markers

The putative cell surface targets were examined by enzymatic treatment to further verify the binding of aptamers to SCLC cell surface markers. After brief treatment of cells with trypsin or proteinase K, diminished binding of aptamers to SCLC cells was observed by flow cytometry in both cases. The same trend was observed using confocal microscopy, only limited amount of fluorescent aptamers getting retained on enzyme treated cell surfaces. These results suggest that selected aptamers indeed bind to cell membrane target molecules, and these SCLC cell surface markers can be affected by protease.

### Example 4

### Validation of Aptamers with Different Cancer Cells and Assay Formats

Before testing with clinical samples, the applicability of developed aptamer probes to other cultured SCLC cell lines was assessed (i.e., to validate the target molecules of developed aptamers as exclusive markers for SCLC). The panel of aptamers showed consistent binding pattern to NCI-H146 and NCI-H128 (Table 2), two SCLC cell lines that have similar cell characteristics as NCI-H69 (target cell used in cell-SELEX).

In contrast to SCLC, three NSCLC cells lines including adenocarcinoma, squamous cell carcinoma, and large cell carcinoma (the one used as control cell in cell-SELEX) did not respond to the selected aptamers except one case (aptamer HCH07 bound to NCI-H23) (Table 2). Moreover, other cancer types including two leukemia cell lines and two liver cancer cell lines were not recognized by these aptamers in most cases (Table 2). Advantageously, the aptamer that bound to adenocarcinoma NCI-H23 is also able to recognize liver cancer cell lines.

TABLE 2
Tests of Developed Aptamers with Cultured Cancer Cell Lines

| Cultured cancer cell lines | Receptors | HCA12 | HCC03 | HCH07 | HCH01 |
|---|---|---|---|---|---|
| NCI-H69 (small cell carcinoma) | IGF II | + | + | + | + |
| NCI-H146 (small cell carcinoma, bone marrow) | IGF II | −− | + | + | + |
| NCI-H128 (small cell carcinoma, pleural effusion) | N/A | + | + | + | + |
| NCI-H661 (large cell carcinoma, lymph node) | N/A | −− | −− | −− | −− |
| NCI-H23 (adenocarcinoma) | PDGF; TGF; EGF | −− | −− | + | −− |
| NCI-H1385 (squamous cell carcinoma, lymph node) | N/A | −− | −− | −− | −− |
| CCRF-CEM (T cell acute lymphoblastic leukemia) | N/A | −− | −− | −− | −− |
| Ramos (B cell human Burkitt's lymphoma) | N/A | −− | −− | −− | −− |
| IMEA (liver cancer) | N/A | −− | −− | + | −− |
| BNL (liver cancer) | N/A | −− | −− | + | −− |

In addition to the tests with live cancer cells, the aptamers developed from live cells can also recognize fixed cells, which is the main assay format for retrospective analysis of preserved specimens such as sputum and biopsy in early detection studies. Formalin-fixed, paraffin-embedded cell line tissue arrays from SCLC and NSCLC samples were processed. After incubation with fluorescent dye labeled aptamers, washing, and dehydration, stained array slides were mounted for array scanning and confocal imaging. Binding of aptamer probes was found to be specific to SCLC as only background level binding existed for NSCLC.

Notably, most aptamers bound to the periphery of target cells as shown in a magnified confocal microscopy image. This binding pattern is similar to that observed in tests of live cells, and further confirmed that aptamers indeed bind to their target molecules on fixed cells. These data indicate that specific recognition of cell line tissue array by aptamers is dependent on the presence of cell surface markers, which are still biochemically active after fixing cells.

### Example 5

### Clinical Sample Tests and Detection of SCLC Cells in Whole Blood Samples

The sensitivity and specificity of the aptamers were assessed for their ability to detect cancer cells in clinical sample from SCLC patients. Substantial change in fluorescence intensity was noted in the SCLC patient's sample after incubation with dye-labeled aptamers, indicating that aptamers developed for cultured cells are also able to recognize the cancer cells from SCLC patients. This result demonstrates the applicability of these aptamers to clinical samples, one important prerequisite for successful detection of SCLC patient cells in complex biological matrix by the aptamers.

In addition, whether aptamers retain the ability to specifically recognize SCLC cells in the presence of human blood environment was evaluated. The binding of fluorescently labeled aptamers to SCLC cells mixed with human whole blood was assessed by flow cytometry. As controls, aptamers were also tested with human whole blood and SCLC cells in buffer. The aptamers' specificity in human whole blood was consistent with those obtained in buffer experiments. SCLC cells were recognized by aptamers specifically, and no interference from various blood cells was observed. For the stability of aptamers in human blood environment, it has been found that modification of aptamers with non-natural nucleic acids can significantly improve the half-life while they still sustain the binding ability.

### Example 6

### Extraction and Detection of SCLC Cells with Aptamer-Conjugated Nanoparticles

During the early stage of lung cancer, malignant lesions begin to shed circulating cells. To evaluate the potential of the selected aptamers for early lung cancer detection, aptamer-conjugated magnetic nanoparticles and aptamer-conjugated fluorescent nanoparticles were prepared. The materials were used to isolate, enrich, and detect rare SCLC cells. The spiked tumor cells were first incubated with aptamer conjugated magnetic and fluorescent nanoparticles. Magnetic nanoparticle bound cells were then isolated by magnetic separation. After recovery, the fluorescence of the dye-doped nanoparticles were measured, which also bound to the isolated cells through aptamer. Whereas two different types of SCLC cells were effectively isolated and detected, the extraction of NSCLC cells was very inefficient. Additionally, very low background fluorescence signal was observed in the control experiment using DNA library conjugated nanoparticles, suggesting that nonspecific extraction of tumor cells is rare with this method.

Effective enrichment and detection of SCLC cells were verified by confocal imaging results, which showed that the extracted tumor cells were indeed binding to aptamer-conjugated nanoparticles. Moreover, the dye-doped nanoparticles confer great sensitivity to the detection of extracted rare tumor cells. Therefore, this aptamers-conjugated nanoparticle approach demonstrated its capability to enrich and detect rare lung cancer cells, which is critical for early diagnosis of lung cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An aptamer that specifically binds a lung cancer cell.

2. The aptamer of claim 1, wherein the aptamer binds to small lung cancer cells with greater affinity than to non-small lung cancer cells.

3. The aptamer claim 1, wherein the aptamer comprises a polynucleotide comprising the nucleic acid sequence of SEQ ID NO:1.

4. The aptamer claim 1, wherein the aptamer comprises a polynucleotide comprising the nucleic acid sequence of SEQ ID NO:2.

5. The aptamer claim 1, wherein the aptamer comprises a polynucleotide comprising the nucleic acid sequence of SEQ ID NO:3.

6. The aptamer claim 1, wherein the aptamer comprises a polynucleotide comprising the nucleic acid sequence of SEQ ID NO:4.

7. The aptamer claim 1, wherein the aptamer comprises a polynucleotide comprising the nucleic acid sequence of SEQ ID NO:5.

8. The aptamer of claim 1, wherein the aptamer is conjugated to a detectable label.

9. The aptamer of claim 8, wherein the detectable label is a fluorophore.

10. The aptamer of claim 8, wherein the aptamer is a radioisotope.

11. The aptamer of claim 1, wherein the aptamer is conjugated to a nanoparticle.

12. A method of detecting a lung cancer cell in a biological sample, the method comprising the steps of:
(a) providing a biological sample comprising a lung cancer cell;
(b) contacting the biological sample with an aptamer that selectively binds the lung cancer cell; and
(c) detecting the aptamer bound to the lung cancer cell.

13. The method of claim 12, wherein the biological sample is blood.

14. The method of claim 12, wherein the biological sample is sputum.

15. The method of claim 12, wherein the aptamer binds to small lung cancer cells with greater affinity than to non-small lung cancer cells.

16. The method of claim 12, wherein the aptamer comprises a polynucleotide comprising a nucleic acid sequence selected from the group consisting of: SEQ ID NO:1, SEQ ID NO:2, SEQ ID NO:3, SEQ ID NO:4, and SEQ ID NO:5.

17. A method comprising the steps of:
(a) providing a single-stranded DNA library comprising at least one million single-stranded DNA molecules having unique nucleic acid sequences;
(b) providing a first sample of small cell lung cancer cells;
(c) mixing the library with the first sample under conditions which allow binding of some of the DNA molecules in the library to the small cell lung cancer cells;
(d) separating the DNA molecules that bind to the small cell lung cancer cells from the DNA molecules that do not bind to the small cell lung cancer cells;
(e) mixing the separated DNA molecules that bind to the small cell lung cancer cells with a first sample of non-small cell lung cancer cells under conditions which allow binding of some of the separated DNA molecules that bind to the small cell lung cancer cells to the non-small cell lung cancer cells; and
(f) separating the DNA molecules that do not bind to the non-small cell lung cancer cells from the DNA molecules that do bind to the non-small cell lung cancer cells; and
(g) collecting the DNA molecules that do not bind to the non-small cell lung cancer cells.

18. The method of claim 17, further comprising the steps of:
(h) mixing the collected DNA molecules that do not bind to the non-small cell lung cancer cells with a second sample of small cell lung cancer cells under conditions which allow binding of some of the collect DNA molecules that do not bind to the non-small cell lung cancer cells to the small cell lung cancer cells;
(i) separating the DNA molecules that bind to the small cell lung cancer cells in step (h) from the DNA molecules that do not bind to the small cell lung cancer cells;
(j) mixing the separated DNA molecules that bind to the small cell lung cancer cells of step (i) with a second sample of non-small cell lung cancer cells under conditions which allow binding of some of the separated DNA molecules that bind to the small cell lung cancer cells of step (h) to the non-small cell lung cancer cells in the second sample; and
(k) separating the DNA molecules that do not bind to the non-small cell lung cancer cells in step (j) from the DNA molecules that do bind to the non-small cell lung cancer cells; and
(l) collecting the DNA molecules that do not bind to the non-small cell lung cancer cells from step (k).
